(19) United States
(12) Patent Application Publication
(10) Pub. No.: US 2017/0100822 A1
(43) Pub. Date: Apr. 13, 2017

(54) POWER TORQUE LIMITING ADAPTER

(71) Applicant: Brian James Cutler, Rowland Heights, CA (US)

(72) Inventor: Brian James Cutler, Rowland Heights, CA (US)

(21) Appl. No.: 15/290,247

(22) Filed: Oct. 11, 2016

Related U.S. Application Data
(60) Provisional application No. 62/239,151, filed on Oct. 8, 2015.

(51) Int. Cl. B25B 23/147; A61B 90/00; A61B 17/70; A61B 17/88; F16D 7/04; B25B 23/00

(52) U.S. Cl. CPC B25B 23/147; F16D 7/044; B25B 23/0035; A61B 17/7082; A61B 17/8875; A61B 90/03; A61B 2090/031

(57) ABSTRACT

A torque limiting adapter readily usable with rotary drivers and tool holders customarily received in a chuck of rotary drivers. The torque limiting adapter is installed in the rotary driver, and in turn, receives the tool holder which holds a fastener or other item to be rotatably driven into the substrate. The torque limiting adapter includes an input shaft received by the rotary driver, a separate output shaft, and an internal spring clutch adjustable to transmit only a desired torque value from the input shaft to the output shaft. A rotatable adjuster adjusts a spring of the spring clutch to vary maximum transmissible torque. The rotatable adjuster includes a locking mechanism to maintain a selected maximum torque value.

### REFERENCE TO RELATED APPLICATION

This application claims priority to U.S. Provisional Utility Application Ser. No. 62/239,151, filed Oct. 8, 2015.

### FIELD OF THE DISCLOSURE

The present disclosure relates to torque transmitting implements, and more particularly, to a self-limiting torque transmitting implement to be installed between a powered rotary driver and a tool to be rotated by the powered rotary driver. The self-limiting torque transmitting implement limits torque which can be imposed on the tool to be rotated.

### BACKGROUND

Tools have been developed to drive rotary fasteners and other items into supporting substrates. If no provision is made to limit torque imposed on the rotary fasteners, they may be overtightened and destroy threading that would otherwise retain them in place. In particular, rotary drivers such as powered drills, and rotatable tool holders which are typically secured within chucks of the rotary drivers are frequently made with no provision for torque limitation. There exists a need for a device which may be readily assembled with rotary drivers and associated tool holders to limit torques applied to the fasteners and other driven items. Beyond this, there exists a need for a torque limiting device having provision for adjusting the amount of torque which may be transmitted to the fastener and other driven items.

### SUMMARY

The disclosed concepts address the above stated situation by providing a torque limiting adapter readily usable with rotary drivers and tool holders customarily received in a chuck of rotary drivers. The torque limiting adapter is installed in the rotary driver, and in turn, receives the tool holder which holds a fastener or other item to be rotatably driven into the substrate. The torque limiting adapter includes an input shaft received by the rotary driver, a separate output shaft, and an internal spring clutch adjustable to transmit only a desired torque value from the input shaft to the output shaft.

A rotatable adjuster adjusts a spring of the spring clutch to vary maximum transmissible torque. The rotatable adjuster includes a locking mechanism to maintain a selected maximum torque value.

It is an object to provide improved elements and arrangements thereof by apparatus for the purposes described which is inexpensive, dependable, and fully effective in accomplishing its intended purposes.

### BRIEF DESCRIPTION OF THE DRAWINGS

Various objects, features, and attendant advantages of the disclosed concepts will become more fully appreciated as the same becomes better understood when considered in conjunction with the accompanying drawings, in which like reference characters designate the same or similar parts throughout the several views, and wherein:

FIG. 1 is a longitudinal cross sectional view of a torque limiting adapter, taken along line A-A of FIG. 3, according to at least one aspect of the disclosure;

FIG. 2 is a redundant view of FIG. 1, taken along line B-B of FIG. 1, provided to avoid unnecessary clutter of reference numerals and their leader lines;

FIG. 3 is an environmental side view of the torque limiting adapter of FIG. 1;

FIG. 4 is a cross sectional view of the torque limiting adapter of FIG. 1, taken along line C-C in FIG. 1;

FIG. 5 is a top view of the torque limiting adapter of FIG. 1; and

FIG. 6 is a cross sectional view of the torque limiting adapter of FIG. 1, taken along line D-D in FIG. 2.

### DETAILED DESCRIPTION

Referring first to FIGS. 2 and 3, according to at least one aspect of the disclosure, there is shown a torque limiting adapter 100 for interfacing between a powered rotary driver (represented by its chuck 102 in FIG. 3) and a driven tool 104 (FIG. 3). Torque limiting adapter 100 may comprise an outer housing 22 comprising a top wall 210 including an inlet opening 30, an opposed bottom wall 31 including an outlet opening 32, and a lateral wall 33 spanning top wall 210 and bottom wall 31. A chamber 34 is surrounded by top wall 210, bottom wall 31, and lateral wall 33.

An input shaft 13 extends through inlet opening 30 of top wall 210. Input shaft 13 terminates in a flange 16 of transverse dimension greater than a transverse dimension of input shaft 13. Flange 16 is contained within outer housing 22.

An output shaft 35 is axially aligned with input shaft 13. Output shaft 35 extends through outlet opening 32 of outer housing 22.

A spring clutch 14 is adjustable to transmit torques imposed on input shaft 13 to output shaft 35, and to establish disengagement of input shaft 13 from output shaft 35. Spring clutch 14 may further comprise an upper member 36, an axially aligned lower member 18, a clutch spring 15 clamping upper member 36 against lower member 18, and an adjuster 37 adjustable to vary clamping pressure exerted by clutch spring 15.

Adjuster 37 may comprise a threaded adjuster member 1 threadedly engaging a threaded interior surface 38 of outer housing 22 and arranged to travel axially along output shaft 35 and to bear against clutch spring 15 when rotated. A locking mechanism 2 is configured to maintain threaded adjuster member 1 in a selected adjustment position along output shaft 35 relative to clutch spring 15.

A receiver 20 including a socket 310 is configured to receive and rotate driven tool 104 responsive to rotation of output shaft 35. Receiver 20 may comprise a drive tool lock 40 configured to releasably retain driven tool 104 in socket 310.

It should be noted at this point that orientational terms such as upper, lower, top, and bottom refer to the subject drawing as viewed by an observer. The drawing figures depict their subject matter in orientations of normal use, which could obviously change with changes in body posture and position. Therefore, orientational terms must be understood to provide semantic basis for purposes of description only, and do not imply that their subject matter can be used only in one position.

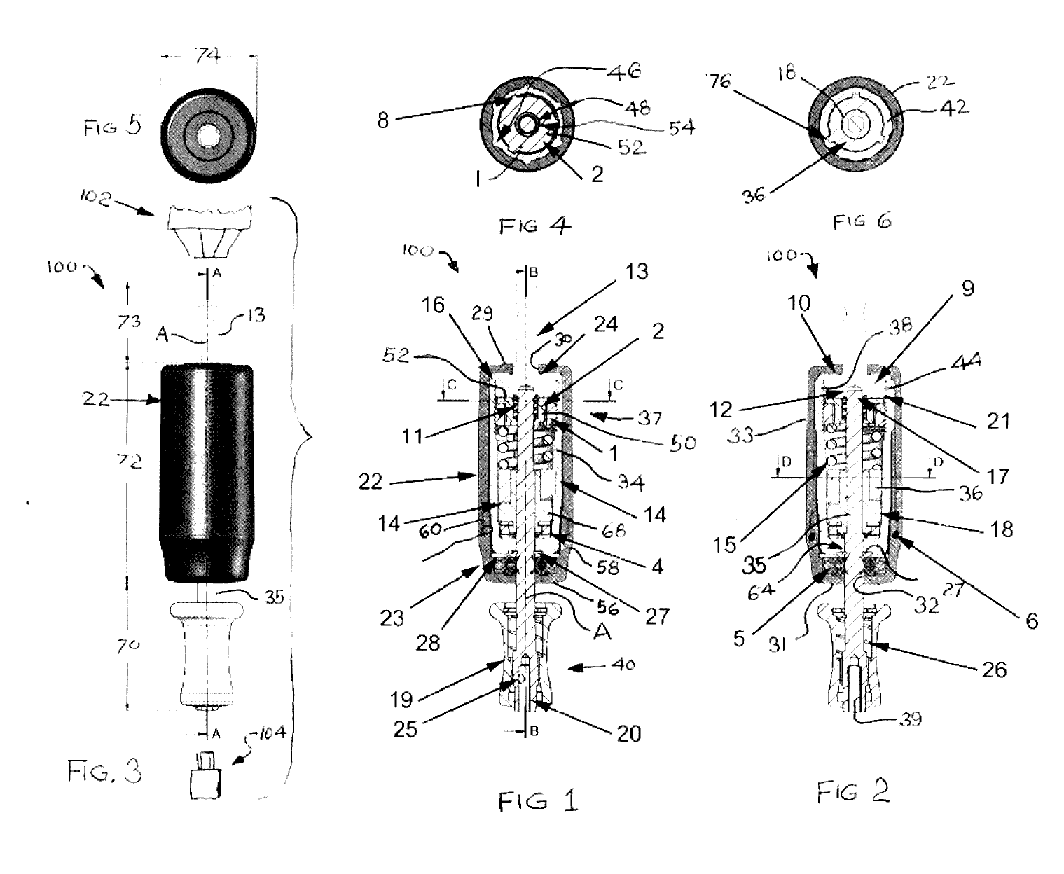

References to an axis, including axial alignment and axial travel as examples, refer to longitudinal axis A shown in FIG. 1.

As will now be explained, outer housing 22 may be complemented by an interior housing 42. In the absence of interior housing 42, threaded interior surface 38 would be formed as part of outer housing 22. Accordingly, outer housing 33 may comprise interior housing 42 including internal threads 44 threadably engaging corresponding threads 21 of the enlarged head 10 of input shaft 13, and axially extending internal grooves 46 (FIG. 4). Threaded adjuster member 1 engages internal threads 44 of interior housing 42.

Locking mechanism 2 may comprise internal adjustment member splines 48 (FIG. 4) on threaded adjuster member 1, and a locking nut 50 (called out in FIG. 1) including an outwardly projecting locking nut flange 52 (FIG. 1). Locking nut 50 includes external splines 54 (FIG. 4) engageable with internal adjustment member splines 48(star). This constrains threaded adjuster member 1 from rotation relative to outer housing 22. Locking nut 50 also includes outwardly projecting tabs 8 on locking nut flange 52 configured to interfit with axially extending internal grooves 46 of interior housing 42. An adjuster spring 11 urges locking nut 50 into engagement with threaded adjuster member 1.

Locking nut 50 has the appearance of an inverted brimmed cylindrical hat, when considering locking nut flange 52, as seen in the cross section of FIG. 1.

Outer housing 22 may be formed in two mating sections, to facilitate fabrication using methods other than three dimensional printing. To this end, outer housing 22 may further comprise a lower housing section 23 including a floor 56, a lateral wall 58, and exterior threads 60 on lateral wall 58. Outer housing 22 may comprise corresponding outer housing threads 62 enabling lower housing section 23 to thread to corresponding outer housing threads 62 of outer housing 22, and to close over components contained within chamber 34 of outer housing 22.

Drive tool lock 40 may comprise a detent ball 25. Receiver 20 may comprise an axially slidable sleeve 110 slidably arranged selectively to press detent ball 25 into engagement with driven tool 104 (FIG. 3), and to release detent ball 25 from engagement with driven tool 104.

Torque limiting adapter 100 may further comprise seals sealing chamber 34 of outer housing 22 against infiltration by contaminants These seals may comprise an upper O-ring seal 24 at inlet opening 30, and a lower O-ring seal 6 between outer housing 22 and lower housing section 23, and a lip seal assembly 5 at outlet opening 32.

Output shaft 35 may include a circumferential groove 64 (FIG. 2), and a clip ring 27 engaging circumferential groove 64. Clip ring 27 has a transverse dimension greater than that of outlet opening 32 of outer housing 22. Clip ring 27 is further entrapped by a thrust washer 28 between clip ring 27 and lip seal assembly 5. Output shaft 35 is thus constrained from sliding out of outlet opening 32 and being lost.

Torque limiting adapter 100 may further comprise a bearing 4 between lower member 18 of spring clutch 14 and floor 68 of interior housing 42.

In use, adjuster member 1 is rotated, and due to outer threads 9 engaging the same threads 38 engaged by enlarged head 10 of input shaft 13, adjuster member 1 travels axially relative to outer housing 22. This influences spring pressure of spring clutch 14. After adjuster member 1 has been adjusted as desired, locking mechanism 2 is activated to maintain the selected torque value arising from the above adjustment.

It will be appreciated that enlarged head 10 of input shaft 13 is tightened to a greater torque (when contacting top wall 29 of outer housing 22) than the highest untightening torque anticipated in the use of torque limiting adapter 100.

In an exemplary usage, torque limiting adapter 100 is used to install pedicle screws (not shown) in surgery, and may have the following dimensions. Dimension 70 may be forty millimeters; dimension 72 may be about sixty-nine or seventy millimeters; dimension 73 may be about twenty-five or twenty-six millimeters; and dimension 74 may be thirty millimeters.

FIG. 6 shows details of spring clutch 14. Notably, upper member 36 is constrained against rotation relative to interior housing 42 by a spline and groove arrangement 76. Also, it is seen that lower member 18 may have a hexagonal passage 78 to cooperate with hexagonal outer configuration of output shaft 35.

FIG. 2 shows that an upper end 17 of output shaft 35 is received within a pilot hole 12 of enlarged head 10 of input shaft 13 to help maintain concentricity of output shaft 35. This portion of output shaft 35 is rotatable relative to input shaft 13. That is, it would have a smooth surface, or alternatively, if hexagonal, would be of dimensions smaller than those of pilot hole 12.

While the disclosed concepts have been described in connection with what is considered the most practical and preferred implementation, it is to be understood that the disclosed concepts are not to be limited to the disclosed arrangements, but are intended to cover various arrangements which are included within the spirit and scope of the broadest possible interpretation of the appended claims so as to encompass all modifications and equivalent arrangements which are possible.

I claim:

1. A torque limiting adapter for interfacing between a powered rotary driver and a driven tool, comprising:
an outer housing comprising a top wall including an inlet opening, an opposed bottom wall including an outlet opening, and a lateral wall spanning the top wall and the bottom wall, and a chamber surrounded by the top wall, the bottom wall, and the lateral wall;
an input shaft extending through the inlet opening of the top wall, the input shaft terminating in a flange of transverse dimension greater than a transverse dimension of the input shaft, the flange contained within the outer housing;
an output shaft axially aligned with the input shaft, the output shaft extending through the outlet opening of the outer housing;
a spring clutch adjustable to transmit torques imposed on the input shaft to the output shaft and to establish disengagement of the input shaft from the output shaft, wherein the spring clutch further comprises an upper member, an axially aligned lower member, a clutch spring clamping the upper member against the lower member, and an adjuster adjustable to vary clamping pressure exerted by the clutch spring, the adjuster comprising a threaded adjuster member threadedly engaging a threaded interior surface of the outer housing and arranged to travel axially along the output shaft and to bear against the clutch spring when rotated;
a locking mechanism configured to maintain the threaded adjuster member in a selected adjustment position along the output shaft relative to the clutch spring; and
a receiver including a socket configured to receive and rotate the driven tool responsive to rotation of the output shaft, the receiver comprising a drive tool lock configured to releasably retain the driven tool in the socket.

2. The torque limiting adapter of claim 1, wherein the outer housing comprises an interior housing including:
internal threads threadably engaging corresponding threads of the flange of the input shaft; and
axially extending internal grooves.

3. The torque limiting adapter of claim 2, wherein the threaded adjuster member engages the internal threads of the interior housing.

4. The torque limiting adapter of claim 2, wherein the locking mechanism comprises:
internal adjustment member splines on the threaded adjuster member;
a locking nut including an outwardly projecting locking nut flange, the locking nut including external splines engageable with the internal adjustment member splines, thereby constraining the threaded adjuster member from rotation relative to the outer housing, and outwardly projecting tabs on the locking nut flange configured to interfit with the axially extending internal splines of the interior housing; and
an adjuster spring urging the locking nut into engagement with the threaded adjuster member.

5. The torque limiting adapter of claim 2, further comprising seals sealing the chamber of the outer housing against infiltration by contaminants

6. The torque limiting adapter of claim 5, wherein the seals comprise an upper O-ring seal at the inlet opening, and a lower O-ring seal between the outer housing and the lower housing section, and a lip seal assembly at the outlet opening

7. The torque limiting adapter of claim 1, wherein the outer housing further comprises a lower housing section including a floor, a lateral wall, and exterior threads on the lateral wall, and the outer housing comprises corresponding outer housing threads enabling the lower housing section to thread to the corresponding outer housing threads of the outer housing and to close over components contained within the chamber of the outer housing.

8. The torque limiting adapter of claim 7, further comprising a bearing between the lower member of the spring clutch and the floor of the interior housing.

9. The torque limiting adapter of claim 1, wherein
the drive tool lock comprises a detent ball; and
the receiver comprises an axially slidable sleeve slidably arranged selectively to press the detent ball into engagement with the driven tool, and to release the detent ball from engagement with the driven tool.

10. The torque limiting adapter of claim 1, wherein the output shaft includes a circumferential groove, and a clip ring engaging the circumferential groove, the clip ring having a transverse dimension greater than the outlet opening of the outer housing, whereby the output shaft is constrained from sliding out of the outlet opening and being lost.
